(19) United States
(12) Patent Application Publication
(10) Pub. No.: US 2026/0137743 A1
(43) Pub. Date: May 21, 2026

(54) METHODS OF RELIEVING PAIN

(71) Applicant: Infinitum Health, LLC, Phoenix, AZ (US)

(72) Inventor: Kevin Thomas Engholdt, Phoenix, AZ (US)

(21) Appl. No.: 19/301,642

(22) Filed: Aug. 15, 2025

Related U.S. Application Data
(63) Continuation of application No. PCT/US2024/015646, filed on Feb. 13, 2024.
(60) Provisional application No. 63/485,439, filed on Feb. 16, 2023.

(51) Int. Cl. A61K 36/185; A61K 9/00; A61K 31/714; A61K 33/08; A61K 36/28; A61K 36/76; A61K 38/48; A61P 25/06; A61P 29/00

(52) U.S. Cl. CPC A61K 36/185; A61K 9/0053; A61K 31/714; A61K 33/08; A61K 36/28; A61K 36/76; A61K 38/4873; A61P 25/06; A61P 29/00; C12Y 304/22002

(57) ABSTRACT

Provided herein are methods and compositions for reducing and preventing pain. In some embodiments, the methods include administration of a composition to an individual experiencing pain, the composition including one or more of Incarvillea sinensis, Salix alba, Tanacetum parthenium, Petasites hybridus, papain, and bromelain.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application n is a continuation of PCT Application No. PCT/US2024/015646, filed Feb. 13, 2024, and claims priority to U.S. Provisional Patent Application No. 63/485,439, filed Feb. 16, 2023, which is hereby incorporated by reference in its entirety.

### BACKGROUND

The present disclosure relates generally to the field of pharmaceuticals and herbal supplements. More particularly, the present disclosure relates to combinations of herbal and other chemical ingredients useful in relieving pain.

According to the CDC, approximately 1 in 5 Americans (about 50 million people) suffer from chronic pain. Of these, nearly 20 million Americans report that the chronic pain interferes with their daily lives. Acute pain is one of the most common complaints that lead Americans to access the health care system, resulting in more than 115 million emergency department visits each year. Migraines affect an estimated 14% of the global population, and an estimated 12% of the United States population. Severe headaches and migraines together affect approximately 15% of the U.S. population.

Many available pain-relieving treatments include opioids, which may cause dependencies. Opioid dependency has contributed to an opioid epidemic. As a result, there is a movement to avoid the use of opioids in managing pain. Additionally, long-term use of current pain-relieving drugs may cause health complications. Therefore, there is a need for novel methods of reducing and preventing pain that do not cause such dependencies and complications.

### SUMMARY

Disclosed herein are methods and compositions for reducing pain in an individual. In some embodiments, the method includes identifying an individual having pain, and administering to the individual a composition. In some embodiments, the composition includes effective amounts of Incarvillea sinensis, Salix alba, Tanacetum parthenium, and papain.

In some embodiments, administering includes administration on an as-needed dosage basis. In some embodiments, the pain is acute pain. In some embodiments, the pain is chronic pain. In some embodiments, the pain is headache pain. In some embodiments, the composition further includes an effective amount of Petasites hybridus. In some embodiments, the composition further includes an effective amount of bromelain.

In some embodiments, administering includes oral administration. In some embodiments, the composition is formulated as a tablet or capsule. In some embodiments, the composition includes about 500 mg of Incarvillea sinensis; about 250 mg of Salix alba; about 200 mg of Tanacetum parthenium; about 100 mg of Petasites hybridus; about 450 mg of papain; and about 450 mg of bromelain.

Further provided herein are compositions for reducing frequency or intensity of pain in an individual. In some embodiments, the composition includes effective amounts of Incarvillea sinensis, Salix alba, Tanacetum parthenium, and Petasites hybridus.

In some embodiments, the composition further comprises an effective amount of papain. In some embodiments, the composition further comprises an effective amount of bromelain. In some embodiments, administering includes oral administration. In some embodiments, the composition is formulated as a tablet or capsule.

In some embodiments, the composition includes about 300-700 mg of Incarvillea sinensis; about 10-400 mg of Salix alba; about 10-400 mg of Tanacetum parthenium; about 10-300 mg of Petasites hybridus; about 200-700 mg of papain; and about 200-700 mg of bromelain. In some embodiments, the composition further includes about 10-200 mg of magnesium. In some embodiments, the composition further includes about 10-200 mcg of vitamin B-12.

In some embodiments, the composition includes about 500 mg of Incarvillea sinensis, about 250 mg of Salix alba, about 200 mg of Tanacetum parthenium, about 100 mg of Petasites hybridus, about 450 mg of papain, and about 450 mg of bromelain. In some embodiments, the composition further comprises about 50 mg of magnesium and about 50 mcg of vitamin B-12.

Also provided herein are methods of reducing pain in an individual. In some embodiments, the method includes administering to an individual having pain a composition. In some embodiments, the composition includes effective amounts of Incarvillea sinensis, Salix alba, Tanacetum parthenium, and Petasites hybridus. In some embodiments, administering includes administration on a daily or twice-daily basis.

In some embodiments, the frequency or intensity of the pain is reduced in the individual. In some embodiments, the pain is acute pain. In some embodiments, the pain is chronic pain. In some embodiments, the pain is headache pain.

In some embodiments, the composition further comprises an effective amount of papain. In some embodiments, the composition further comprises an effective amount of bromelain. In some embodiments, administering includes oral administration. In some embodiments, the composition is formulated as a tablet or capsule. In some embodiments, the composition includes about 500 mg of Incarvillea sinensis, about 250 mg of Salix alba, about 200 mg of Tanacetum parthenium, about 100 mg of Petasites hybridus, about 450 mg of papain, and about 450 mg of bromelain.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIG. 1 depicts the chemical structure of incarvillateine, a compound found in Incarvillea sinensis.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the drawings, can be arranged, substituted, combined, separated, and designed in a wide variety of different configurations, all of which are explicitly contemplated herein.

As summarized herein, aspects of methods for relieving and preventing pain such as short term (acute), long term (chronic), and headache types of pain by the administration of compositions of the present disclosure are provided herein. Further disclosed herein are compositions that may be used for reducing or prevent pain in an individual.

It is to be understood that this disclosure is not limited to particular embodiments described, as such may, of course, vary. It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Unless defined otherwise, technical and scientific terms used herein have the same meaning as commonly understood by one of ordinary skill in the art to which the present disclosure belongs. For purposes of the present disclosure, the following terms are defined below.

“Inhibit,” “inhibiting,” and “inhibition” and/or “reduce,” “reducing,” and “reduction” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90; 100%, or any amount of reduction in between the specifically recited percentages, as compared to native or control levels.

A “subject,” “patient,” or “individual,” as used herein, includes any animal that experiences pain. Suitable subjects (“patients”) include laboratory animals (such as mouse, rat, rabbit, or guinea pig), farm animals, and domestic animals or pets (such as a cat or dog). Non-human primates and, preferably, human patients, are included.

Provided herein are methods of reducing or preventing pain, including short term (acute), long term (chronic), and headache types of pain, by administration of compositions described herein. Further described herein are methods of producing analgesia in an individual by administration of compositions described herein. In some embodiments, the compositions include an effective amount of one or more naturally derived ingredients selected from Incarvillea sinensis, Tanacetum parthenium, Salix alba, Petasites hybridus, papain, and bromelain. Such ingredients may be included as an essence or extract of the stated ingredient. For example, Incarvillea sinensis may be included as extract of Incarvillea sinensis. In some embodiments, administration of the compositions can advantageously reduce or prevent pain without causing a dependency in an individual.

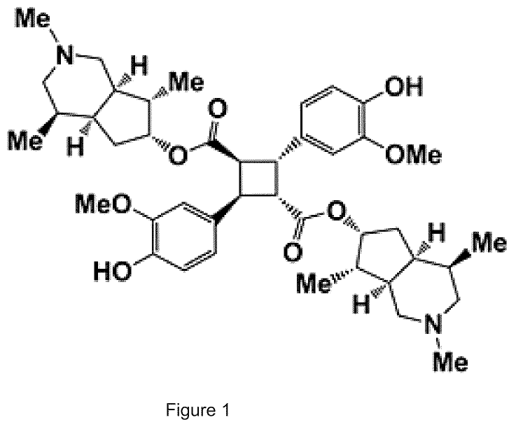

In some embodiments, Incarvillea sinensis is included in the compositions described herein. Incarvillea sinensis is a plant of the family Bignoniaceae. Incarvillateine isolated from Incarvillea sinensis has demonstrated significant analgesic (pain relief) activity. Incarvillateine has the chemical structure shown in FIG. 1. Incarvillea sinensis has historically been used in China and Japan in teas for rheumatism for over 5000 years. Without being bound by theory, recent research has suggested that incarvillateine's mechanism of action involves inhibiting fatty acid binding proteins (FABPs) in endocannabinoid signaling pathways. (Berger, 2012). In some embodiments, the composition includes an effective amount of Incarvillea sinensis. In some embodiments, the composition comprises between 300-700 mg of Incarvillea sinensis, between 400-600 mg of Incarvillea sinensis, between 450-550 mg of Incarvillea sinensis, between 480-520 mg of Incarvillea sinensis, about 500 mg of Incarvillea sinensis, or a value within any of the aforementioned ranges, or a range constructed from any of the aforementioned values.

In some embodiments, Salix alba is included in the compositions described herein. Willow bark has been used as a traditional medicine for more than 3500 years. Willow Bark (Salix alba) contains salicin, which is chemically similar to aspirin (acetylsalicylic acid). Prior to the discovery of aspirin, salicin was investigated as an anti-inflammatory and antipyretic. (Desborough and Keeling, 2017). In some embodiments, the composition includes an effective amount of Salix alba. In some embodiments, the composition comprises between 10-400 mg of Salix alba, between 100-350 mg of Salix alba, between 150-300 mg of Salix alba, between 180-270 mg of Salix alba, about 200 mg of Salix alba, about 250 mg of Salix alba, or a value within any of the aforementioned ranges, or a range constructed from any of the aforementioned values.

In some embodiments, Tanacetum parthenium is included in the compositions described herein. Tanacetum parthenium, known as feverfew, or bachelor buttons, is a flowering plant in the daisy family, Asteraceae. It is a traditional medicinal herb that has been used to prevent migraine headaches. The active principals probably include one or more of the sesquiterpene lactones known to be present, including parthenolide, flavonoid glycosides, and pinenes. (Pareek, 2011). In some embodiments, the composition includes an effective amount of Tanacetum parthenium. In some embodiments, the composition comprises between 10-400 mg of Tanacetum parthenium, between 100-300 mg of Tanacetum parthenium, between 150-250 mg of Tanacetum parthenium, between 180-220 mg of Tanacetum parthenium, about 200 mg of Tanacetum parthenium, or a value within any of the aforementioned ranges, or a range constructed from any of the aforementioned values.

In some embodiments, Petasites hybridus may be included in the compositions described herein. Petasites hybridus is a perennial shrub whose root (butterbur) was used for medicinal purposes in ancient times and has been rediscovered since the middle of the last century for clinical applications including migraine. Petasites hybridus root (butterbur) has been shown to be an effective preventive treatment for migraine. (Lipton, 2004). In some embodiments, the composition includes an effective amount of Petasites hybridus. In some embodiments, the composition comprises between 10-300 mg of Petasites hybridus, between 15-200 mg of Petasites hybridus, between 50-150 mg of Petasites hybridus, between 80-120 mg of Petasites hybridus, about 100 mg of Petasites hybridus, or a value within any of the aforementioned ranges, or a range constructed from any of the aforementioned values.

In some embodiments, papain is included in the compositions described herein. Papain, derived from papaya, has been used as an anti-inflammatory (Devaraj, 2020; Menon and Muthusekhar, 2021) and as an analgesic (Devaraj, 2020; Acharya, 2016; Yaidikar, 2019). Papain has also been shown to be useful as a wound cleanser (Ajlia, 2010). In some embodiments, the composition includes an effective amount of papain. In some embodiments, the composition comprises between 200-700 mg of papain, between 300-600 mg of papain, between 350-550 mg of papain, between 400-500 mg of papain, about 450 mg of papain, or a value within any of the aforementioned ranges, or a range constructed from any of the aforementioned values.

In some embodiments, bromelain is included in the compositions described herein. Bromelain is a proteolytic enzyme found in pineapple. Bromelain has been used to alleviate inflammation (Jayachandran, 2017; Dighe, 2010; Brien, 2004), pain (Dighe, 2010; Brien, 2004; Acharya, 2016), and joint stiffness (Dighe, 2010). Other therapeutic benefits of bromelain include inhibition of platelet aggregation, relief of sinusitis, amelioration of surgical traumas, relief of thrombophlebitis, relief of pyelonephritis, relief of angina pectoris, enhancement of the absorption of drugs, relief of bronchitis, and relief of osteoarthritis, as bromelain has been shown to have fibrinolytic, anti-edematous, anti-inflammatory, anti-cancer, anti-diarrhea, and wound healing properties (López, 2017; Hirche et al., 2020; Wickham et al., 2019; Hikisz and Bernasinska-Slomczewska, 2021). In some embodiments, the composition includes an effective amount of bromelain. In some embodiments, the composition comprises between 200-700 mg of bromelain, between 300-600 mg of bromelain, between 350-550 mg of bromelain, between 400-500 mg of bromelain, about 450 mg of bromelain, or a value within any of the aforementioned ranges, or a range constructed from any of the aforementioned values.

In some embodiments, the composition further includes magnesium. Without being bound by theory, magnesium may aid feverfew (Tanacetum parthenium) to be better absorbed in the blood stream and aid in headache relief. The magnesium may be included as any pharmaceutically acceptable salt of magnesium. In one embodiment, magnesium is included as magnesium oxide. In some embodiments, the composition includes an effective amount of magnesium. In some embodiments, the composition comprises between 10-200 mg of magnesium, between 15-100 mg of magnesium, between 20-80 mg of magnesium, between 35-75 mg of magnesium, about 50 mg of magnesium, or a value within any of the aforementioned ranges, or a range constructed from any of the aforementioned values.

In some embodiments, the composition further includes vitamin B-12. In some embodiments, vitamin B-12 is included as methylcobalamin (MeCbl). In some embodiments, vitamin B-12 is included as cyanocobalamin. Without being bound by theory, vitamin B-12 may aid feverfew (Tanacetum parthenium) to be better absorbed in the blood stream and aid in headache relief. In some embodiments, the composition includes an effective amount of vitamin B-12. In some embodiments, the composition comprises between 10-200 mcg of vitamin B-12, between 15-100 mcg of vitamin B-12, between 20-80 mcg of vitamin B-12, between 35-75 mcg of vitamin B-12, about 50 mcg of vitamin B-12, or a value within any of the aforementioned ranges, or a range constructed from any of the aforementioned values.

Provided herein are methods of reducing pain in an individual. Further provided herein are methods of preventing pain in an individual. In some embodiments, the methods include identifying an individual having pain. In some embodiments, identifying an individual having pain includes identifying an individual currently experiencing pain. In some embodiments, identifying an individual having pain includes identifying an individual with a history of recurring pain, such as chronic pain or migraines.

In some embodiments, the methods further include administering to the individual a composition. In some embodiments, the composition includes effective amounts of Incarvillea sinensis, Salix alba, and Tanacetum parthenium. In some embodiments, the composition further includes an effective amount of papain. In some embodiments, the composition further includes an effective amount of Petasites hybridus, such as Petasites hybridus root (also known as butterbur). In some embodiments, the composition further includes an effective amount of bromelain. In some embodiments, the composition includes an effective amount of magnesium. In some embodiments, the composition includes an effective amount of vitamin B-12.

In some embodiments, the frequency or intensity of the pain is reduced in the individual. In some embodiments, the pain is acute pain. In some embodiments, the pain is chronic pain. In some embodiments, the pain is headache pain (including migraines). In some embodiments, the subject suffers from pain. The pain can come from headaches, stomachaches, cancer, injury, autoimmune disease, or a genetic disorder. In some embodiments, the pain is derived from a systemic issues such as pain arising from joints, muscles, organs, and other sites of inflammation.

In some embodiments, administering includes administration on an as-needed dosage (ad hoc) basis. For example, a composition may be administered when a subject begins to experience a pain or when the pain level increases past a threshold of tolerability for the subject. In other embodiments, administration is preventative and happens before the subject begins to experience pain. In some embodiments, administration comprises weekly administration. In some embodiments, administration comprises daily administration. In some embodiments, administration comprises twice daily administration.

In some embodiments, administering includes oral administration. In some embodiments, the composition is formulated as a tablet. In some embodiments, the composition is formulated as a capsule.

In some embodiments, the composition includes about 300-700 mg of Incarvillea sinensis, about 10-400 mg of Salix alba, about 10-400 mg of Tanacetum parthenium, about 10-300 mg of Petasites hybridus, about 200-700 mg of papain, and about 200-700 mg of bromelain. In some embodiments, the composition includes about 10-200 mg of magnesium. In some embodiments, the composition includes about 10-200 mcg of vitamin B-12.

In some embodiments, the composition includes about 500 mg of Incarvillea sinensis, about 250 mg of Salix alba, about 200 mg of Tanacetum parthenium, about 100 mg of Petasites hybridus, about 450 mg of papain, and about 450 mg of bromelain. In some embodiments, the composition includes about 50 mg of magnesium. In some embodiments, the composition includes about 50 mcg of vitamin B-12.

An exemplary composition for use in the methods presented herein is described below in Table 1.

TABLE 1

| | Amount (mg, unless |
|---|---|
| Ingredient | specified otherwise) |
| Incarvillea sinensis | 500 |
| Willow bark (Salix alba) | 250 |
| Feverfew (Tanacetum parthenium) | 200 |
| Butterbur (Petasites hybridus) | 100 |
| Papain (from Papaya) | 450 |
| Bromelain (from Pineapple) | 450 |
| Vitamin B12 (as Methylcobalamin) | 50 mcg (microgram) |
| Magnesium (as Magnesium Oxide) | 50 |

A second exemplary composition for use in the methods presented herein is described below in Table 2.

TABLE 2

| | Amount (mg, unless |
|---|---|
| Ingredient | specified otherwise) |
| Incarvillea sinensis | 500 |
| Willow bark (Salix alba) | 200 |
| Feverfew (Tanacetum parthenium) | 200 |
| Vitamin B12 (as Methylcobalamin) | 50 mcg (microgram) |
| Magnesium (as Magnesium Oxide) | 50 |

### Methods of Identifying and Measuring Pain

In some embodiments, methods described herein include a step of identifying an individual having a pain. In some embodiments, identifying includes self-identification, for example, a patient identifies herself or himself as having a pain. Having a pain may include currently experiencing a pain or having a known pattern of chronic pain. In some embodiments, a second individual identifies a first individual as having a pain.

In some embodiments, pain may be measured using a pain scale. In some embodiments, the pain scale is unidimensional. In some embodiments, the pain scale is a numeric rating scale, for example, from 0 to 10, with 0 representing no pain and 10 representing the worst possible pain. In some embodiments, the pain scale is a visual analog scale, such as a line representing a spectrum, labeled “no pain” at one end and “worst possible pain” at the other end.

In some embodiments, pain is measured using a categorical scale. For example, in some embodiments, an individual describes the intensity of their pain using descriptors ranging from no pain, mild pain, moderate pain, severe pain, very severe pain, to worst possible pain.

In some embodiments, pain may be measured using a multidimensional tool. In some embodiments, pain is measured using a brief pain inventory. In some embodiments, pain is measured using the McGill pain questionnaire.

In some embodiments, methods of reducing pain described herein may reduce pain as measured by any one of the pain scales or pain measurement tools described herein. For example, pain may be reduced from an 8 to a 2 on a numeric rating scale. For example, pain may be reduced from severe to mild on a categorical scale. For example, pain may be reduced from a score of 60 to a score of 13 according to the McGill pain questionnaire.

In some embodiments, identifying an individual having a pain includes identifying an individual having a pain with a measurement over a certain threshold of a pain scale. For example, identifying an individual having pain may include identifying an individual having severe or greater pain, identifying an individual having pain greater than 7/10, or identifying an individual having pain greater than 50 on the McGill pain questionnaire.

### Pharmaceutical Compositions

In some embodiments, the active ingredients and mixtures of active ingredients may be used, for example, in pharmaceutical compositions comprising a pharmaceutically acceptable carrier prepared for storage and subsequent administration. Also, some embodiments include use of the above-described active ingredients with a pharmaceutically acceptable carrier or diluent. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes and even flavoring agents may be provided in the pharmaceutical composition. For example, sodium benzoate, ascorbic acid and esters of p-hydroxybenzoic acid may be added as preservatives. In addition, antioxidants and suspending agents may be used.

Compositions of the active ingredients may be formulated and used as tablets, capsules, or elixirs for oral administration; suppositories for rectal administration; sterile solutions, suspensions for injectable administration; patches for transdermal administration, and sub-dermal deposits and the like. Injectables can be prepared. in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. If desired, absorption enhancing preparations (for example, liposomes), may be utilized.

For injection, agents may be formulated in aqueous solutions, preferably in physiologically compatible buffers such as Hanks' solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the ingredients herein disclosed for the practice into dosages suitable for systemic administration is within the scope of the disclosure. With proper choice of carrier and suitable manufacturing practice, the compositions disclosed herein, in particular, those formulated as solutions, may be administered parenterally, such as by intravenous injection. The active ingredients can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a patient to be treated.

Pharmaceutical formulations for parenteral administration include aqueous solutions of the active ingredients in water-soluble form. Additionally, suspensions of the active ingredients may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the ingredients to allow for the preparation of highly concentrated solutions.

Pharmaceutical preparations for oral use can be obtained by combining the active ingredients with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active ingredient doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active ingredient doses. Such formulations can be made using methods known in the art. See, for example, U.S. Pat. No. 5,733,888 (injectable compositions); U.S. Pat. No. 5,726,181 (poorly water soluble compounds); U.S. Pat. No. 5,707,641 (therapeutically active proteins or peptides); U.S. Pat. No. 5,667,809 (lipophilic agents); U.S. Pat. No. 5,576,012 (solubilizing polymeric agents); U.S. Pat. No. 5,707,615 (anti-viral formulations); U.S. Pat. No. 5,683,676 (particulate medicaments); U.S. Pat. No. 5,654,286 (topical formulations); U.S. Pat. No. 5,688,529 (oral suspensions); U.S. Pat. No. 5,445,829 (extended release formulations); U.S. Pat. No. 5,653,987 (liquid formulations); U.S. Pat. No. 5,641,515 (controlled release formulations) and U.S. Pat. No. 5,601,845 (spheroid formulations); all of which are incorporated herein by reference in their entireties. The pharmaceutical compositions may be manufactured in a manner that is itself known, for example, by means of conventional mixing, dissolving, granulating, dragee-making, levitating, emulsifying, encapsulating, entrapping, or lyophilizing processes.

To formulate the dosage including one or more active ingredients disclosed herein, known surface active agents, excipients, smoothing agents, suspension agents and pharmaceutically acceptable film-forming substances and coating assistants, and the like may be used. Preferably alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents. In addition to the foregoing ingredients, sweeteners, fragrances, colorants, preservatives and the like may be added to the administered formulation of the compound, particularly when the compound is to be administered orally.

Further disclosed herein are various pharmaceutical compositions well known in the pharmaceutical art for uses that include intraocular, intranasal, and intraauricular delivery. Pharmaceutical formulations include aqueous ophthalmic solutions of the active ingredients in water-soluble form, such as eyedrops, or in gellan gum (Shedden et al., Clin. Ther., 23 (3): 440-50 (2001)) or hydrogels (Mayer et al., Ophthalmologica, 210 (2): 101-3 (1996)); ophthalmic ointments; ophthalmic suspensions, such as microparticulates, drug-containing small polymeric particles that are suspended in a liquid carrier medium (Joshi, A., J. Ocul. Pharmacol., 10 (1): 29-45 (1994)), lipid-soluble formulations (Alm et al., Prog. Clin. Biol. Res., 312:447-58 (1989)), and microspheres (Mordenti, Toxicol. Sci., 52 (1): 101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions may also include drops and sprays often prepared to simulate in many respects nasal secretions to ensure maintenance of normal ciliary action. As disclosed in Remington's Pharmaceutical Sciences, 18th Ed., Mack Publishing Co., Easton, Pa. (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the car. Common solvents for such aural formulations include glycerin and water.

The compositions described herein may be administered by either oral or a non-oral pathways. When administered orally, compositions can be administered in capsule, tablet, granule, spray, syrup, or other such form. Compositions also may be brewed, as with a tea, or formed by dissolving a powdered composition into a fluid, typically water, fruit or vegetable juice, or milk. When administered non-orally, it can be administered as an aqueous suspension, an oily preparation or the like or as a drip, suppository, salve, ointment or the like, when administered via injection, subcutaneously, interperitoneally, intravenously, intramuscularly, or the like. Similarly, it may be administered topically, rectally, or vaginally, as deemed appropriate by those of skill in the art for bringing the ingredients into optimal contact with living tissue.

Agents intended to be administered intracellularly may be administered using techniques well known to those of ordinary skill in the art. For example, such agents may be encapsulated into liposomes, then administered by any of the methods described herein. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. Additionally, due to their hydrophobicity, small organic molecules may be directly administered intracellularly.

In some embodiments, the compositions described herein are formulated into a single pill or tablet. In some embodiments, the pill or tablet has a mass from 10 mg to 3000 mg. In some embodiments, the pill or tablet has a mass from 100 mg to 2500 mg. In some embodiments, the pill or tablet has a mass from 500 mg to 2300 mg. In some embodiments, the pill or tablet has a mass from 800 mg to 2000 mg.

### Methods of Administration

Some embodiments also encompass methods for making and for administering the disclosed compositions. Such disclosed methods include, among others, (a) administration though oral pathways, which administration includes administration in capsule, tablet, granule, spray, syrup, or other such forms; (b) administration through non-oral pathways, which administration includes administration as an aqueous suspension, an oily preparation or the like or as a drip, suppository, salve, ointment or the like; administration via injection, subcutaneously, intraperitoneally, intravenously, intramuscularly, intradermally, or the like; as well as (c) administration topically, (d) administration rectally, or (c) administration vaginally, as deemed appropriate by those of skill in the art for bringing the compound into contact with living tissue; and (f) administration via controlled released formulations, depot formulations, and infusion pump delivery. As further examples of such modes of administration and as further disclosure of modes of administration, disclosed herein are various methods for administration of the disclosed compositions including modes of administration through intraocular, intranasal, and intraauricular pathways.

The pharmaceutically effective amount of the ingredients disclosed herein required as a dose will depend on the route of administration and the physical characteristics of the specific human under consideration. The dose can be tailored to achieve a desired effect, but will depend on such factors as weight, diet, concurrent medication and other factors, which those skilled in the medical arts will recognize.

In practicing the methods, the products or compositions can be used alone or in combination with one another or in combination with other therapeutic or diagnostic agents. These products can be utilized in vivo, ordinarily in a mammal, preferably in a human, or in vitro. In employing them in vivo, the products or compositions can be administered to the mammal in a variety of ways, including parenterally, intravenously, subcutaneously, intramuscularly, colonically, rectally, vaginally, nasally or intraperitoneally, employing a variety of dosage forms. Such methods may also be applied to testing chemical activity in vivo.

As will be readily apparent to one skilled in the art, the useful in vivo dosage to be administered and the particular mode of administration will vary depending upon the age, weight and mammalian species treated, the particular ingredients employed, and the specific use for which these ingredients are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods. In non-human animal studies, applications of potential products are commenced at higher dosage levels, with dosage being decreased until the desired effect is no longer achieved or adverse side effects disappear.

The dosage of active ingredient(s) may range broadly, depending upon the desired affects and the therapeutic indication. Typically, dosages of active ingredient(s) may be between about 10 microgram/kg and 100 mg/kg body weight, preferably between about 100 microgram/kg and 10 mg/kg body weight. In some embodiments, the dosage of active ingredient(s) may be 1, 2, 3, 4, 5, 6, 7, 8 or 9 mg/kg body weight. Alternatively, dosages may be based and calculated upon the surface area of the patient, as understood by those of skill in the art. In a preferred embodiment, administration is oral on a daily, twice daily, or ad hoc basis.

The exact formulation, route of administration and dosage can be chosen in view of the consumer's condition. See for example, Fingl et al., in The Pharmacological Basis of Therapeutics, 1975, which is incorporated herein by reference in its entirety. The magnitude of an administrated dose may vary with the severity of a particular medical or physical condition and the route of administration. The severity of a condition may, for example, be evaluated, in part, by standard prognostic evaluation methods. Further, the dose and perhaps dose frequency, may also vary according to the age, body weight, and response of the individual. A program comparable to that discussed above may be used in veterinary medicine.

A variety of techniques for formulation and administration may be found in Remington's Pharmaceutical Sciences, 18th Ed., Mack Publishing Co., Easton, Pa. (1990), which is incorporated herein by reference in its entirety. Suitable administration routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

The combined active ingredients in the compositions disclosed herein may be orally or non-orally administered to a human patient in the amount of about 0.0007 mg/day to about 7,000 mg/day of the total active ingredients, and more preferably about 0.07 mg/day to about 70 mg/day of the total active ingredients at, one time per day or in other embodiments, over two to about ten times per day. Alternatively, the active ingredients disclosed herein may be administered in the stated amounts continuously by, for example, an intravenous drip. Thus, for a patient weighing 70 kilograms, the preferred daily dose of the total active ingredients would be about 0.0007 mg/kg/day to about 35 mg/kg/day, and more preferable, 0.007 mg/kg/day to about 15 mg/kg/day. Nonetheless, as will be understood by those of skill in the art, in certain situations it may be necessary to administer the active ingredients disclosed herein in amounts that excess, or even far exceed, the above-stated, preferred dosage range to treat effectively and aggressively a desired condition or characteristic.

Ingredients disclosed herein can be evaluated for efficacy and toxicity using known methods. For example, the toxicology of a particular compound or ingredient, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds or ingredients in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including the conditions abated by the compounds or ingredients disclosed herein, including obesity. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound or ingredient in humans.

The active ingredients described above may be used alone or in combination with one another, or in combination with other therapeutic or diagnostic agents. These products can be utilized in vivo or in vitro. The useful dosages and the most useful modes of administration will vary depending upon the age and weight of the consumer, the particular ingredients employed, and the specific use for which these ingredients are employed. In some embodiments, the composition described herein is formulated as a tablet. In some embodiments, the composition described herein is formulated as a capsule. In some embodiments, the composition comprises gelatin. In some embodiments, the composition comprises cellulose. In some embodiments, the composition comprises magnesium stearate.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1

The composition described in Table 2 was administered orally to a 55-year-old male subject suffering from a chronic knee pain. The subject was employed as a construction worker prior to retiring and had previously managed pain with ibuprofen or other non-steroidal inflammatory (NSAID) agents. The composition was administered orally to the subject. The subject stated that within 2 hours, his pain was gone in his knee. The subject also reported that referred pain from his knee that became headaches over time also was reduced to nothing. The subject continued to take said composition for 3 more weeks and pain resolved such that the subject was able to take walks with dog, something he was previously unable to do. The subject has not reported dependencies or toxicities from the composition.

### Example 2

The composition described in Table 2 was administered orally to a 23-year-old female subject who frequently suffered from chronic migraine headaches. The composition was formulated as a capsule and administration was on a daily basis. The subject had been using Excedrin® for years to manage pain. Patient reported that after 3 hours, her migraine pain was completely reduced and remained gone for the remainder of the day. The subject continued to take the composition orally each morning, within headache pain significantly reduced to minor levels, for the next 2 weeks. After two weeks of daily administration, the subject reported that headaches had ceased and administration of the composition stopped. After 2 weeks of no migraines, the subject reported that due to the reduction and prevention of the migraines, she experienced fewer interruptions in her life and greater clarity of thought. Over the next few years, the subject took the composition ad hoc for general pain(s) but has experienced a dramatically greater quality of life due to the lack of migraines. The subject has not reported dependencies or toxicities from the composition.

### Example 3

The composition described in Table 2 was administered orally to a 28-year-old male subject suffering from a chronic ankle pain due to injuries sustained during college football. The subject had previously managed pain with ibuprofen and cortisone shots. 2 hours after administration of the composition, the subject's pain was gone and stayed gone for multiple hours. Pain returned after several hours, and the subject began taking said composition twice daily, once in morning and once in the evening. Pain stayed reduced to nothing during the period of twice daily administration. The subject stopped administration for 1 day and reported that the pain returned by that evening. The subject continued administration twice daily from that point on and has been pain free since. The subject has not reported dependencies or toxicities from the composition.

### Example 4

A composition with a formulation described in Table 1 is administered orally to a subject suffering from chronic pain. Within four hours after administration, the subject reports experiencing the chronic pain is reduced from a 44 to an 8 on the McGill Pain scale.

### Example 5

A compositions with a formulation described in Table 1 is administered orally to a subject suffering from a migraine. Within two hours after administration, the subject reports the migraine pain has lessened from an 8 to a 2 on a numerical pain intensity scale.

Headings are included herein for reference and to aid in locating various sections. These headings are not intended to limit the scope of the concepts described with respect thereto. Such concepts may have applicability throughout the entire specification.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

In the foregoing description, specific details are given to provide a thorough understanding of the examples. However, it will be understood by one of ordinary skill in the art that the examples may be practiced without these specific details.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the claims.

The methods disclosed herein comprise one or more steps or actions for achieving the described method. The method steps and/or actions may be interchanged with one another without departing from the scope of the present disclosure. In other words, unless a specific order of steps or actions is required for proper operation of the method that is being described, the order and/or use of specific steps and/or actions may be modified without departing from the scope of the present disclosure.

In at least some of the described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural or singular terms herein, those having skill in the art can translate from the plural to the singular or from the singular to the plural as is appropriate to the context or application. The various singular/plural permutations may be expressly set forth herein for sake of clarity.

The embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (such as “a reagent” can mean one or more reagents) unless it is contextually clear either one of the elements or more than one of the elements is described. The term “about” as used herein refers to a value within 10% of the underlying parameter (for example, plus or minus 10%), and use of the term “about” at the beginning of a string of values modifies each of the values (for example, “about 1, 2 and 3” refers to about 1, about 2 and about 3). For example, a weight of “about 100 grams” can include weights between 90 grams and 110 grams. Further, when a listing of values is described herein (for example, about 50%, 60%, 70%, 80%, 85% or 86%) the listing includes all intermediate and fractional values thereof (for example, 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of the embodiments.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

### REFERENCES

Acharya et al., A comparative study of effect on reducing pain, inflammation and side effect of combination of enzymes (bacterial proteases, papain, bromelain, vitamin C and rutin) versus conventional non-steroidal anti-inflammatory drugs (diclofenac) in patients of closed fracture lower end radius coming at orthopaedic department of a tertiary care hospital, Int'l J Basic & Clin. Pharmac., (2016) 5:3:1017-1021.
Ajlia et al., Efficacy of Papain-based Wound Cleanser in Promoting Wound Regeneration, Pakistan Journal of Biological Sciences 13 (12): 596-603, 2010, ISSN 1028-880.
Berger W T, Ralph B P, Kaczocha M, Sun J, Balius T E, et al. (2012) Targeting Fatty Acid Binding Protein (FABP) Anandamide Transporters—A Novel Strategy for Development of Anti-Inflammatory and Anti-Nociceptive Drugs. PLOS ONE 7 (12): e50968. doi: 10.1371/journal.pone.0050968.
Brien et al., Bromelain as a Treatment for Osteoarthritis: a Review of Clinical Studies, Evidence-based Complementary and Alternative Medicine, Vol. 1, Issue 3, doi: 10.1093/ecam/neh035.
Devaraj, Efficacy of papain in reducing soft tissue swelling-A case report, Rwanda Medical Journal Vol. 77 (1); March 2020.
Desborough and Keeling, The aspirin story—from willow to wonder drug, British Journal of Haematology, 2017, 177, 674-683.
Dighe et al., Bromelain A Wonder Supplement: A Review, Pharmacologyonline 1:11-18 (2010).
Hirche et al., Eschar removal by bromelain based enzymatic debridement (Nexobrid®) in burns: European consensus guidelines update, Burns 46 (2020) 782-796.
Hikisz & Bernasinska-Slomczewska, Beneficial Properties of Bromelain, Nutrients 2021, 13, 4313. https://doi.org/10.3390/nu13124313.
Jayachandran and Khobre, Efficacy of Bromelain along with Trypsin, Rutoside Trihydrate Enzymes and Diclofenac Sodium Combination Therapy for the treatment of TMJ Osteoarthritis-A Randomised Clinical Trial, Journal of Clinical and Diagnostic Research. 2017 June, Vol-11 (6): ZC09-ZC11, DOI: 10.7860/JCDR/2017/25771.9964.
Lipton et al., Petasites hybridus root (butterbur) is an effective preventive treatment for migraine, Neurology, 2004 Dec. 28; 63 (12): 22240-4.
López, Debridement Applications of Bromelain: A Complex of Cysteine Proteases from Pineapple, Adv Biotech & Micro 3(5): AIBM.MS.ID.555624 (2017).
Menon and Muthusekhar, Effectiveness of Papain as an Anti-Inflammatory Agent in Patients Undergoing Surgical Removal of Impacted Teeth-A Randomized Double Blinded Clinical Trial, J Res Med Dent Sci, 2021, 9(7): 73-76.
Pareek et al., Feverfew (Tanacetum parthenium L.): A systematic review, Pharmacognosy reviews, January-June 2011, Vol. 5 Issue 9.
Wickham et al., Successful treatment of mixed depth flame burns using enzymatic debridement with Nexobrid™ in a patient with aggressive systemic sclerosis (scleroderma), Scars, Burns & Healing, Volume 5:1-4, DOI: 10.1177/2059513118821563.
Yaidikar et al., Papain, an active constituent of Carica Papaya ameliorates neuropathic pain in rats subjected to sciatic nerve ligation by mitigating oxidative damage and excitotoxicity, Acta Scientific Neurology 2.7 (2019): 02-10.

1. A method of reducing recurring pain, chronic pain, or migraines in an individual, comprising:
administering to the individual a composition comprising effective amounts of:
about 300 mg to about 700 mg of Incarvillea sinensis;
about 10 to about 400 mg of Salix alba;
about 10 to about 400 mg of Tanacetum parthenium;
about 10 to about 200 mg of magnesium;
about 10 to about 200 mcg of vitamin B-12,
wherein administering comprises administration on an as-needed dosage basis.

2. The method of claim 1, wherein the composition further comprises an effective amount of Petasites hybridus.

3. The method of claim 2, wherein the composition further comprises an effective amount of bromelain.

4. The method of claim 3, wherein administering comprises oral administration.

5. The method of claim 4, wherein the composition is formulated as a tablet or capsule.

6. The method of claim 5, wherein the composition comprises:
about 500 mg of Incarvillea sinensis;
about 250 mg of Salix alba;
about 200 mg of Tanacetum parthenium;
about 100 mg of Petasites hybridus; and
about 450 mg of bromelain.

7. (canceled)

8. (canceled)

9. (canceled)

10. A composition for reducing frequency or intensity of recurring pain, chronic pain, or migraines in an individual, comprising:
about 300 mg to about 700 mg of Incarvillea sinensis;
about 10 to about 400 mg of Salix alba;
about 10 to about 400 mg of Tanacetum parthenium; and
about 10 to about 200 mg of magnesium;
about 10 to about 200 mcg of vitamin B-12.

11. The composition of claim 10, wherein the composition further comprises an effective amount of papain.

12. The composition of claim 11, wherein the composition further comprises an effective amount of bromelain.

13. (canceled)

14. The composition of claim 12, wherein the composition comprises:
about 300-700 mg of Incarvillea sinensis;
about 10-400 mg of Salix alba;
about 10-400 mg of Tanacetum parthenium;
about 200-700 mg of papain; and
about 200-700 mg of bromelain.

15. (canceled)

16. (canceled)

17. The composition of claim 14, wherein the composition comprises:
about 500 mg of Incarvillea sinensis;
about 250 mg of Salix alba;
about 200 mg of Tanacetum parthenium;
about 450 mg of papain; and
about 450 mg of bromelain.

18. The composition of claim 17, wherein the composition comprises about 50 mg of magnesium and about 50 mcg of vitamin B-12.

19. A method of reducing recurring pain, chronic pain, or migraines in an individual, comprising:
administering to an individual having pain a composition comprising effective amounts of:
about 300 mg to about 700 mg of Incarvillea sinensis;
about 10 to about 400 mg of Salix alba;
about 10 to about 400 mg of Tanacetum parthenium; and
about 10 to about 200 mg of magnesium;
about 10 to about 200 mcg of vitamin B-12
wherein administering comprises administration on a daily or twice-daily basis, and wherein the frequency or intensity of the pain is reduced in the individual.

20. (canceled)

21. (canceled)

22. (canceled)

23. The method of claim 19, wherein the composition further comprises an effective amount of papain.

24. The method of claim 23, wherein the composition further comprises an effective amount of bromelain.

25. The method of claim 24, wherein administering comprises oral administration.

26. (canceled)

27. The method of claim 25, wherein the composition comprises:
about 500 mg of Incarvillea sinensis;
about 250 mg of Salix alba;
about 200 mg of Tanacetum parthenium;
about 100 mg of Petasites hybridus;
about 450 mg of bromelain.

28. (canceled)

29. (canceled)

30. The method of claim 1, wherein the composition comprises:
about 500 mg of Incarvillea sinensis;
about 200 mg of Salix alba;
about 200 mg of Tanacetum parthenium;
about 50 mcg Vitamin B12; and
about 50 mg of magnesium.

31. The composition of claim 10, wherein the composition comprises:
about 500 mg of Incarvillea sinensis;
about 200 mg of Salix alba;
about 200 mg of Tanacetum parthenium;
about 50 mcg Vitamin B12; and
about 50 mg of magnesium.

32. The method of claim 19, wherein the composition comprises:
about 500 mg of Incarvillea sinensis;
about 200 mg of Salix alba;
about 200 mg of Tanacetum parthenium;
about 50 mcg Vitamin B12; and
about 50 mg of magnesium.
